(19) United States
(12) Patent Application Publication
(10) Pub. No.: US 2011/0104716 A1
(43) Pub. Date: May 5, 2011

(54) NOVEL METHOD FOR SCREENING BRAIN-ACTIVE COMPOUNDS

(76) Inventor: Hasan Mohejeri, Zurich (CH)

(21) Appl. No.: 12/673,427

(22) PCT Filed: Aug. 8, 2008
(86) PCT No.: PCT/EP08/06558

§ 371 Date: Feb. 12, 2010

(30) Foreign Application Priority Data
Aug. 9, 2007 (EP) EP07007053
Jan. 10, 2008 (EP) 08000362.7

(51) Int. Cl. G01N 33/53

(52) U.S. Cl. 435/7.21

(57) ABSTRACT

The invention relates to a novel screening method for brain-active substances and mixtures using hippocampal slices.

### FIELD OF THE INVENTION

The present invention relates to a novel methodology to screen for bioactive compounds or mixtures that affect brain functions and performance by determining if the test compound induces long term potentiation in hippocampal slices. Such brain functions may include, but are not limited to, learning and memory, alertness, mood, coping with stress, with psychotic conditions and with migraine.

### BACKGROUND OF THE INVENTION

Brain functions rely on neuronal circuits and an optimal brain functioning such as mental performance, learning and memory are dependent on synaptic plasticity; i.e. strengthening neuronal connections by the recruitment of new receptors, formation of new synapses and eventually the generation of new neuronal connections.

Long term potentiation (LTP) is the term used to describe the long-lasting enhancement of synaptic transmission (minutes to hours in vitro, days or weeks in vivo) which occurs at particular synapses within the brain following a short, conditioning, burst of presynaptic electric stimulation. LTP can be induced in virtually all brain structures and the induction of LTP is shown to obey similar mechanistic (biochemical) principals in all brain regions, which result in strengthening the active synapses thus leading to facilitated transmission of signals between the neighboring neurons (Huang et al 1994. Learn Mem 1:74-82).

The LTP phenomenon is best studied in connection to modeling learning and memory in vitro. Memory, learning and alertness utilize neuronal circuits in the midbrain, especially in the hippocampus where information is processed and memory is consolidated. The formation of (long-term) memory and the efficient functioning of the brain depend on synthesis of new proteins for the reinforcement of communicative strength between neurons. The production of new proteins devoted to synapse reinforcement is triggered by chemical and electrical signals within neurons.

Hippocampal LTP is widely considered to be one of the major mechanisms by which memories are formed and stored in the brain. Hippocampal LTP has been observed both in vitro and in living animals. Under experimental conditions, applying a series of short, high-frequency electric stimuli to a synapse can potentiate the strength of the chemical synapse for minutes to hours. Most importantly, hippocampal LTP contributes to synaptic plasticity in living animals, providing the foundation for a highly adaptable nervous system.

Two different receptor types are primarily involved in the process of hippocampal LTP, namely the N-methyl-D-aspartate (NMDA) receptor complex and the α-amino-3-hydroxy-5-methyl-4-isoxazole propionic acid (AMPA) receptor. During LTP, the major excitatory neurotransmitter, glutamate, is released from the presynaptic neuron, binds to and activates the AMPA receptor on the postsynaptic membrane, leading to its depolarization. At resting membrane potentials, the NMDA receptor channel is blocked by magnesium ions, but depolarization of the postsynaptic membrane removes this block, enabling NMDA receptor activation and subsequent entry of calcium into the cell. This rise in intracellular calcium is believed to activate protein kinases, leading to gene transcription and the construction of reinforcing proteins (Niehoff (2005), The Language of Life: How Cells Communicate in Health and Disease, 210-223) and resulting in enhanced sensitivity of the AMPA receptor, thus further facilitating neurotransmission and maintenance of LTP.

Historically, the most widely used experimental means of inducing LTP has been to deliver electric tetanic stimulation to the presynaptic axon of a synapse or group of synapses. The frequency of this tetanus is typically 100 Hz, and the duration typically 1 second. Whereas electrical stimulation of brain slices is suitable for studying the mechanisms of LTP and has been validated as a model system for brain functions in vivo, electrical stimulation does not allow screening of brain active substances.

There is an increasing interest in the development of compounds, as well as nutraceutical compositions, which may be used to improve learning, memory and alertness, as mood improvers or to reduce psychosocial stress. These type of compositions would be desirable for administration to: the elderly, young people, individuals who need especially high memory and attention in their daily work (such as students, construction workers, drivers, pilots, physicians, salespeople, executives, housewives, and “high performance professionals”) and people who are under mental or daily stress as well as persons who are prone to psychiatric instability or stress, such as schizophrenia or depression.

Thus, a compound or nutraceutical composition which enhances LTP in general and in particular hippocampal LTP, would improve learning, memory, alertness, mood, and would lead generally to stress reduction, improved ability to cope with psychosocial burden and improved brain function and wellbeing.

Brain slice cultures have been used in the past for various screening tools. See for example, Sundstrom et al 2005 Drug Discovery Today 10 (14):993-1000. However, these assays mimic neurodegeneration by observing dying cells and the ability of test compounds to prevent cell death.

### DETAILED DESCRIPTION OF THE INVENTION

This invention relates to a method for screening the brain-active substances that are able to induce LTP in brain slices (i.e. in vitro), and provides a method for validation of the observed LTP effect on brain function in vivo.

This invention thus relates to an assay to determine if a test substance modulates brain functions in vivo comprising the steps of:
a) incubating hippocampal slices from an animal with the test substance for a time sufficient for the test substance to potentially interact with NMDA and/or AMPA receptors present in the hippocampal slices to induce Long Term Potentiation (LTP); and
b) determining if LTP induction occurred in the brain slices, wherein a positive result demonstrates the test substance's ability to induce LTP in vitro, and is indicative of its ability to improve brain functions in vivo.

In preferred embodiments, the induction of LTP is detected by immunochemical staining of biochemical markers of LTP. Known biochemical markers of LTP include:
expression of the activated form of the cAMP response element binding protein (phosphorylated CREB),
expression of the activated form of the mitogen-activated protein kinase (phosphorylated MAPK), and
changes in the levels of AMPA receptors (AMPA rec) present on the cell surface.

Theses and similar markers can be detected and quantified by means of immunohistochemical staining of the slice cultures using commercially available antibodies including phospho-CERB: (UPSTATE No. 05-807), phospho-MAPK: (CELL SIGNALING No. 4376S), and AMPA receptor: (UPSTATE No. 07-660).

Experimental systems for induction of LTP can be set up using mammalian brain sections of both sexes and varying ages (postnatal day 5 to adult age) including rats, mice and guinea pigs. Preferably, rats or mice are used.

To measure the activation of these markers, hippocampal slices are generally prepared as is known in the art (Stoppini et al. 1991 J Neurosci Methods 37(2):173-82, Scanziani et al. 1992 Neuron 9(5):919-27). They are then incubated with the extracts or pure compounds to be tested for LTP-induction activity for the time necessary to induce LTP (typically between a few minutes to one hour).

Sections are then washed extensively and fixed with, for example, 2% paraformaldehyde solution and stained with the antibodies according to the manufacturers' instructions. Quantification of the activation can be performed either by blinded observes at 100 to 400× magnification or by the use of automated fluorescence imaging software such as the system sold by Cellomics, Pittsburgh, Pa.

Alternatively, expression levels of LTP markers may be determined by other known methods, such as reverse transcriptase polymerase chain reaction (RT-PCR), enzyme linked immunosorbent essay (ELISA) or multiplex measurement technologies.

Improved Brain Functions by this Invention

Throughout this specification and claims, the term “improved brain functions” is meant to refer to the conditions of supporting and maintaining brain wellness and balance, such as:
Enhanced learning, including:
   language processing
   problem solving
   intellectual functioning
Ability to cope with psychosocial burdens
Enhanced attention and concentration
Enhanced memory and the capacity for remembering, especially short-term memory
Enhanced mental alertness and mental vigilance, reduction of mental fatigue
Stabilization of mental status including:
   Relieving post-partum conditions
   Relieving psychological burden due to separation of partners, children, death of beloved people or due to marital problems
   Relieving problems associated with change of domicile, work, and similar conditions
   Relieving stressful conditions following an traffic accident and other negative social pressure
Stress relief, including:
   treatment, prevention and alleviation of symptoms related to work overload, exhaustion and/or burn out
   increased resistance or tolerance to stress
   favoring and facilitating relaxation in normal healthy individuals
“Condition improvement”, including
   reducing irritability and tiredness
   reducing, preventing or alleviating physical and mental fatigue
   promoting good-quality sleep, that is to act against insomnia and sleep disorders and to increase energy in more general terms, in diseased or normal healthy individuals
   conditions related to reduced neurotransmission

In a preferred aspect of the present invention the compositions may be used as nutritional supplements, particularly for people who may feel a need for enhanced cognitive function and/or psychosocial support. A non-exhaustive list of people who would benefit from enhanced cognitive function would include:
elderly people,
students or persons who are preparing for exams,
children who are engaged in a great deal of learning, i.e. infants, toddlers, pre-school children and school children,
construction workers, or those operating potentially dangerous machinery,
truck drivers, pilots, train drivers, or other transportation professionals,
air traffic controllers,
salespeople, executives, and other “high performance professionals”,
police officers and military personnel, fire fighters,
housewives,
   or for anyone exposed to high amounts of stress in their daily work or who needs especially high attention/concentration/and high mental and psychological performance in their daily work, such as those participating in sports, chess players, golfers, professional performers (actors, musicians and the like), or for anyone experiencing social stress, such as after divorce, traffic accidents, change of domicile or work or after losing beloved people.

Aside from applications for humans, this invention has additional uses in the veterinary world. Animals which can benefit from enhanced brain function include those animals which are subject to stressful conditions. Such conditions occur, for example, after capture or transport or may be due to housing conditions, due to change of domicile or owner, when the animals develop analogous disorders and are distressed or aggressive, or display stereotypic behavior, or anxiety and obsessive-compulsive behavior. Animals which are subject to stress would also include those which are racing animals (e.g. dogs, horses, camels), or used in various sports, performing animals (such as circus animals and those appearing on stage, television or in the movies) and horses which perform dressage and other highly disciplined routines.

Preferred “animals” are pets or companion animals and farm animals. Examples of pets are dogs, cats, birds, aquarium fish, guinea pigs, (jack) rabbits, hares and ferrets. Examples of farm animals are aquaculture fish, pigs, horses, ruminants (cattle, sheep and goats) and poultry.

The following non-limiting Examples are presented to better illustrate the invention.

### Example 1

### Preparation and Composition of a Thyme Extract

Dried leaves of thyme were milled and extracted with supercritical carbon dioxide. The parameters of extraction were as follows: temperature of 45° C.; working pressure: 300 bar (-to) or 100 bar (-se); 17 kg (-to) and 15 kg (-se) of carbon dioxide per 1 kg of plant material were needed; the extracts were obtained in the separator by throttling the pressure to 60 bar at 30° C. 25 kg (-to) or 50 kg (-se) of plant material respectively yielded 1 kg of extract.

A typical thyme CO₂ extract disclosed by this invention had the following composition (analyzed by Gas Chromatography):

The total content of essential oil was 65.3% (the remaining parts are plant waxes). Volatile components are listed below:

| | |
|---|---|
| Thymol | 53% |
| P-Cymene | 34% |
| Linalool | 2.2% |
| Caryophyllene | 2% |
| Carvacrol | 1.7% |

### Example 2

### Preparation and Composition of an Oregano Extract

Dried leaves of Oregano were milled and extracted with supercritical carbon dioxide. The parameters of extraction were as follows: temperature of 45° C.; Working pressure: 300 bar (-to) or 100 bar (-se); 17 kg (-to) and 15 kg (-se) of carbon dioxide per 1 kg of plant material were needed. The extracts were obtained in the separator by throttling the pressure to 60 bar at 30° C. 25 kg (-to) or 50 kg (-se) of plant material respectively yielded 1 kg of extract.

Oregano extract had the following composition (analyzed by Gas Chromatography): The total content of essential oil of a typical oregano extract used in this invention was 80-95% (the remaining parts are plant waxes). Major volatile components are as follow:

| | |
|---|---|
| Carvacrol | 60-90% |
| Thymoquinone | 2-8% |
| P-Cymene | less than 10% |
| Thymol | less than 10% |
| Linalool | less than 10% |
| Caryophyllene | less than 10% |

### Example 3

### Hippocampal Slice Cultures and Induction of LTP

Seven-day-old Wistar rats were decapitated using a guillotine. In less than 1 minute the skull was opened, the cerebral hemispheres were separated and transferred and both hippocampi were dissected and transferred into ice cold buffer containing 137 mM NaCl, 5 mM KCl, 0.85 mM Na₂HPO₄, 1.5 mM CaCl₂, 0.66 mM KH₂PO₄, 0.28 mM MgSO₄, 1 mM MgCl₂, 2.7 mM NaHCO₃, 1 mM Kynurenic acid and 0.6% D-glucose.

Transversal hippocampal slices (typically 400 μm) were prepared using a vibrating blade microtome (VT1200S; Leica Microsystems (Schweiz) AG, Heerbrugg, Switzerland) in the same buffer. Hippocampal slices were individually placed on a membrane insert (Millicell Culture Plate Inserts, 0.4 μm) and cultivated at 35° C., 5% CO₂, 95% humidity in a medium containing a 1:1 mixture of BME and MEM (both from Invitrogen) containing 25% heat-inactivated horse serum, 1× GlutaMAX, 1× Penicillin/Streptomycin, 0.6% glucose and 1 mM Kynurenic acid (Stoppini et al. 1991 J Neurosci Methods 37(2):173-82).

After 48 h in culture, synaptic NMDA receptors were activated by addition of single extracts or their components for 15 min in 140 mM NaCl, 5 mM KCl, 1.3 mM CaCl₂, 25 mM HEPES (pH 7.3), 33 mM D-glucose and 0.02 mM bicuculline methiodide. Sarcosine (100 μM) and ALX5407 (20 nM) were used routinely as positive controls. An additional positive control comprised the addition of 200 μM glycine to sister cultures.

After the treatments, sections were washed and fixed for immunohistochemistry. Markers of enhanced synaptic activity, normally associated with LTP, representing an in vitro (or ex vivo) model of learning and memory, were quantified (see Table 1, below).

Table 1. Relative activation of synaptic markers after treatment with both extracts and some of their constituent compounds in comparison to sister cultures treated with buffer. The activation of any of these markers (or a combination thereof) is observed in classical LTP experiments. Data are compared to vehicle treated sections, which is set as 100%. Effects of the positive control (glycine) is also shown.

TABLE 1

| | Source | | | |
|---|---|---|---|---|
| Substance | (Cat. no.) | pCREB | pMAPK | AMPA rec. |
| Thyme extract | Flavex | ± | ++ | 320% |
| | (039-002) | | | |
| Oregano extract | Flavex | ± | ± | ± |
| | (103.012) | | | |
| Thymol | Fluka (89330) | ++++ | +++ | 250-767% |
| P-Cymene | Aldrich | ++ | ++++ | ± |
| | (C121452) | | | |
| Linalool | Fluka (62140) | ± | ± | ± |
| Caryophyllene | Fluka (22075) | ± | ± | not done |
| Carvacrol | Aldrich | + | − | 0-176% |
| | (282197) | | | |
| Glycine | Sigma, | ++ | ++ | 123-298% |
| | (G7403) | | | |

While ± demonstrated no change in the activation status, ++++, ++ and − show a qualitative maximal activation, a half-maximum activation and a reduction of activation, respectively. % numbers signify the increase of APMA receptors on the cell surface (all in comparison to corresponding vehicle treated sister cultures.

Treatment of hippocampal cultures with the thyme extract as well as with thymol or p-cemene induced biochemical markers typical for LTP (pCREB: activated form of the cAMP response element binding protein; pMAPK: activated form of the mitogen-activated protein kinase; AMPA rec.: cell surface present AMPA receptor).

Thyme extract, thymol, p-cymene and similar compounds induce activation of biochemical pathway leading to LTP induction, thus can activate hippocampal functions. On the other hand, oregano extract and its major constituent carvacrol, Linalool and Caryophyllene lack the LTP-inducing activity.

### Example 4

### Effects of Thyme and Oregano Extracts in the Acoustic Startle Response Assay, a Model of Non-Associative Learning and Memory in Zebrafish

Habituation is one of the simplest forms of non-associative learning and memory, resulting in the reduction of a response to a repeated stimulus (Thompson et al (1966) Psychol Rev, 73:16-43.). One of the prominent behaviors studied in vertebrates is the startle response, a fast contraction of body muscles caused by a sudden acoustic, tactile or visual stimulus mediated by simple neuronal circuitry (Koch. (1999) Prog Neurobiol, 59: 107-28).

For assessment of the effects of the thyme and oregano extracts on the acoustic startle response (ASR) in zebrafish, 20 days post fertilization (d.p.f) fish, which are known to possess a functional blood-brain-barrier similar to that of mammals, were allowed to swim in a 48 well plate (Millipore, Watford, UK), one fish per well. The fish were exposed to different concentrations of the test compound, as dissolved in their swimming water. 24 h later the fish were placed in the tracking system. An automated live tracking system comprising of a Sony XC E150 CE Camera (Tracksys Ltd., Nottingham, UK) and Ethovision software (Noldus, Wageningen, The Netherlands) was used to monitor the fish. After 15 minutes of habituation the fish were exposed to a sequence of auditory tones synchronized by the Ethovision software. Auditory cues of 0.6 second in length, 200 Hz in frequency and 113 decibels, as measured using an NM102 Noise Meter (NoiseMeter Ltd., Burton Fleming, UK) placed above the 48 well plate, were produced from side-mounted speakers (Bell Packard; placed 10 cm away from the side of the 48 well plate) connected to a Dell computer and given at 1 second intervals (referred to as the inter-trial interval, ITI). An auditory tone session consisted of up to 50 tones, with two sessions being given with 15 minutes recovery period between each episode of auditory stimulation. The ASR was analyzed for each individual fish by measuring the distance moved in response to each auditory stimulus; this provided a quantitative readout of the startle response and was defined as the distance moved by the fish during 1 s from the beginning of the auditory stimulus. Results are shown in Table 2.

Table 2. In two independent experiments the effects of the thyme and oregano extracts were tested on the ASR. Addition of the thyme extract to the fishes' environment affects their cognitive ability over a large concentration range, whereas addition of the oregano extract was ineffective in this learning paradigm. * represents a significant learning difference to age-matched control group exposed only to vehicle and ns signifies a non-significant change of learning behavior.

TABLE 2

| | | |
|---|---|---|
| Concentration | THYME | OREGANO |
| in swimming | Extract | Extract |

| water (mg/ml) | EXP 1 | EXP 2 | EXP | EXP 1 |
|---|---|---|---|---|
| 0.003 | * | * | ns | ns |
| 0.001 | * | ns | ns | ns |
| 0.0003 | * | ns | ns | ns |

This data show that the compound's activity in inducing hippocampal LTP in vitro (shown in Table 1) correlates with the potential to improve the corresponding brain function in vivo (learning behavior) as shown in Table 2.

### Example 5

### Effects of Thyme Extract in a Traditional Rodent Model of Learning and Memory

Associative learning and memory behavior was also examined in rodents after oral administration of thyme extract, which was identified by the ex vivo LTP assay and proved efficacy in the Zebrafish model. To this aim, mice were subjected to an associative learning and memory paradigm. Reaction box bottom was fitted with a 36V electric grid. When animals receive an electric shock, their normal reaction is to jump up onto an insulated platform to avoid the pain stimulus. The majority of animals that jumped back onto the grid, would, upon receiving the electrical shock, rapidly jump back up on the platform. Animals were trained for 5 min, and the number of times each mouse was shocked, or made an error, was noted. This data constituted the learning data. Re-tests were done at 24 and 48 h, with these trials serving as the memory tests. The number of animals shocked in each group, the time prior to jumping down from the platform and the number of errors in the first 3 min were recorded. At five days after conclusion of training, memory decay was tested.

When compared to vehicle-treated littermates (negative control) or mice treated with gingko-biloba or rolipram (positive controls), thyme treated animals exhibited a significant better learning and memory performance during the training and memory phase and after the wash-out period.

These data prove again that the positive ex vivo activity in the chemical LTP test system, as claimed by this invention, predicts a positive outcome of learning and memory testing in vivo.

### Example 6

### Effects of Thyme Extract in New, Totally Automated, Rodent Model of Learning and Memory

We have tested the cognitive performances of mice treated with ginkgo biloba (positive control) and thyme extract and compared them with their vehicle treated age-matched controls in the IntelliCage® system (NewBehavior AG, Zürich, Switzerland) allowing automatic monitoring the animal behavior over an extended period of time in home cages. IntelliCage® was validated for testing experimental animals in cognitive and motivational paradigms (Galsworthy et al. 2005, Behav Brain Res 157: 211-217; Onishchenko et al. 2007, Toxicol Sci 97, 428-437) in social groups without overtly produced stress by social isolation and frequent test environments. Moreover, IntelliCage® system discriminated rapidly between animals with various degree of hippocampal damage housed together with controls (Lipp et al. 2004, FENS annual meeting), indicating that IntelliCage® is suitable for testing hippocampal-dependent behaviour.

### Test Groups and Treatments

The study included 3 test groups (n=12-14 per group). All mice were administered test substances or vehicle via daily oral gavage (10 ml/kg) throughout the 8 week study.

### IntelliCage®

The IntelliCage® is a system which enables automated monitoring of spontaneous and learning behaviour of transponder carrying mice in a homecage-like environment (NewBehavior AG, Zurich, Switzerland). Each IntelliCage® has four recording (operant) chambers. The recording chambers fit into the corners of the cage, each covering a 15×15×21 cm right-angled triangular area of floor space. Each animal is recognized by means of an implanted transponder throughout the entire experiment. In-cage antennae enable automatic monitoring of each individual mouse's corner visits; photo-beams within each corner enable automated recording of individual nosepokes and licks of the water bottle spouts. Four triangular mouse shelters were placed in the centre of the cage, above which was situated a food hopper, enabling ad libitum access to food. All corners are equipped with tubing, through which air-puffs can be delivered as aversive stimulation.

### Object Recognition Test

To test the intrinsic exploratory activity of the groups, two identical objects were placed in 2 of 4 corners of the cages. The animals had the opportunity to explore the cage and had free access to water and feed. Visits to each corner were recorded 3 h before and 3 h after the objects were presented. Control group did not exhibit any change of the visiting pattern, whereas the ginkgo and the thyme treated groups significantly increased the duration of the time visiting the corners with new objects. These results show that thyme-treated animals performed better than the vehicle treated age-matched controls in the object recognition test.

### Place Learning (Measure for Learning Capacity)

In order to investigate place learning behaviour, mice were tested in this module. The least-preferred corner, as determined during the nose-poke adaptation phase, was designated as the “correct” corner for each individual mouse. Only nose-pokes within this corner would trigger opening of the motorised doors and permit access to the water bottle; nose-pokes in all other corners were “incorrect” and resulted in aversive stimulation, in the form of an air-puff (1 s).

Learning curve for thyme extract treated group in comparison with control animals and Ginkgo biloba (GBE) treated animals revealed that all groups learned the task equally well.

### Reversal of Place Learning (Measure of Memory Performance)

In this module, the “correct” corner was designated as that which was diagonally opposite to the “correct” corner of the previous test module. Visits to “incorrect” corners were again subjected to negative reinforcement (an air-puff). As expected, the initial error rate was high at the beginning of this module, but all groups learned quickly the task. The performance of thyme-treated group was significantly better than both other groups (p=0.011): whereas thyme-treated group improved to 20% error rate after 10 h, both vehicle and Ginkgo treated groups exhibited a plateau error rate of around 60-70%.

These data prove once more that the positive ex vivo activity in the chemical LTP test system, as claimed by this invention, predicts a positive outcome of learning and memory testing in vivo.

1. An assay to determine if a test substance modulates brain functions in vivo comprising the steps of:
a) incubating hippocampal slices from an animal with the test substance for a time sufficient for the test substance to potentially interact with NMDA and/or AMPA receptors present in the hippocampal slices to induce Long Term Potentiation (LTP); and
b) determining if LTP induction occurred in the brain slices, wherein a positive result demonstrates the test substance's ability to induce LTP in vitro, and is indicative of its ability to improve brain functions in vivo.

2. An assay according to claim 1 wherein the hippocampal slices are from a mouse, rat or guinea pig.

3. An assay according to claim 1 wherein interaction between the test substance and receptors is measured by detected by immunochemical staining of biochemical markers of LTP.

4. An assay according to claim 1 wherein the biochemical markers are selected from the group consisting of:
expression of pCREB (activated form of cAMP response element binding protein);
expression of pMAPK (activated form of mitogen-activated protein kinase), and
changes in the levels of AMPA rec (cell surface AMPA receptors).

5. An assay according to claim 1 wherein the improved brain function in vivo is selected from the group consisting of: maintaining cognitive wellness and balance, improvement of learning, improvement of language processing, improvement of problem solving, improvement of intellectual functioning, improvement of motivation, improvement of an ability to cope with psychosocial burdens, improvement of attention, improvement of concentration, improvement of memory, improvement of the capacity for remembering, improvement of mental alertness, improvement of mental vigilance, reduction of mental fatigue, stabilization of mental status, improvement of mood, a stress reliever or reducer, a reducer of work overload stress, a reducer of stress-related exhaustion and/or burn out, and improvement of the ability to relax.

6. A method according to claim 1 wherein the test substance is a pure compound or combination of compounds and/or a plant extract.
